(19) United States
(12) Patent Application Publication
(10) Pub. No.: US 2017/0049316 A1
(43) Pub. Date: Feb. 23, 2017

(54) METHOD AND APPARATUS FOR TESTING A PATIENT'S VISUAL FIELD

(71) Applicant: IBISVISION LIMITED, ABERDEEN (GB)

(72) Inventor: BLAIR DONALDSON, ABERDEEN (GB)

(21) Appl. No.: 15/241,724

(22) Filed: Aug. 19, 2016

(30) Foreign Application Priority Data
Aug. 20, 2015 (GB) 1514782.0

(51) Int. Cl. A61B 3/024; A61B 3/00

(52) U.S. Cl. CPC A61B 3/024; A61B 3/0033; A61B 3/0058; A61B 3/0025

(57) ABSTRACT

A method and apparatus for testing a patient's visual field. The testing apparatus comprises a display (3) and a patient input device (9). A home target (19) is presented on the display (3), along with a cursor (21) being moveable under control of the patient input device (9). Once the cursor (21) is moved to the home target (19), a visual field target (11) is presented on the display (3) at a first position. The visual field target (11) is recorded as detected if the cursor (21) moves a predetermined distance towards the visual field target (11). The step is repeated for further visual field targets (11) at further positions for determining the patient's visual field based on the positions of the visual field targets (11) that have been detected.

The present invention relates to a method, apparatus and software for use in testing a patient's visual field.

Visual field tests are examinations conducted in order to determine the extent of a patient's visual field. These tests can be used to detect dysfunctions in a patient's vision, in particular dysfunctions in a patient's peripheral vision, which may be related to medical conditions such as glaucoma, strokes and brain tumours.

There are several variations of peripheral visual field tests, which are usually performed using dedicated test equipment called perimeters which run an automated computer program. This is called Standard Automated Perimetry (SAP). The Humphrey perimeter visual field test is one example. In this test, a white hemispherical bowl is positioned at a set distance in front of a patient, such that when the patient rests their chin on a provided chin rest, each point on the inner surface of the bowl is the same distance from the patient's eye or eyes being tested. The patient looks straight ahead into the centre of the inner surface of the bowl, and fixes their gaze on a fixation target. Visual field targets are then presented at various known positions on the inner surface of the bowl, corresponding to various positions in the patient's peripheral vision. The patient activates a switch when they see a presented visual field target. A computer program records visual field targets as seen if the patient responds and activates the switch, and unseen if the patient does not respond and does not activate the switch.

These visual field test results can then be compiled into a map displaying the area of the visual field tested together with an indication of the quality of vision at the known positions of the presented visual field targets. The map can be stored in a computer database, and used to help with the diagnosis of disease or compared with maps from the patient's previous visual field tests in order to detect changes in the patient's vision.

However, there are several problems associated with SAP tests, such as the Humphrey perimeter test, which can lead to unreliable test results. For example, a patient often finds it difficult to maintain fixation, and concentration, on a fixed target for a prolonged period. Thus, there is a natural tendency for the patient to lose their visual fixation on the fixation target. The patient may then activate the switch and respond to the presentation of a visual field target that would have otherwise registered as not seen if their gaze had been fixed on the fixation target. This response by the patient would be a false positive response.

Furthermore, after losing their visual fixation or concentration on the fixation target, the patient may look around at different points on the hemispherical bowl in an attempt to quickly locate the next visual field target. This again can lead to a false positive response from the patient.

Moreover, the edges of the patient's peripheral vision can become blurred after the patient has had their gaze fixed on the fixation target for a prolonged period. As a consequence, the patient may be unsure as to whether or not they have seen a presented visual field target. The patient may then miss the next presented visual field target whilst deliberating about this. The patient may therefore not activate the switch in response to the presentation of a visual field target that is actually within their visual field. This lack of response would mean that the visual field target is recorded as not seen by the patient. This response would be a false negative response.

In addition, conventional SAP testing machines, such as a Humphrey perimeter, are expensive, highly specialised, and bulky. As a result, obtaining and maintaining SAP testing machines represents a significant overhead for clinical practices, both in terms of cost and storage. Furthermore, the equipment is often so specialised that it cannot be updated as testing methods and protocols evolve over time. As such, equipment can become outdated and redundant, which limits the incentives for clinical practices to invest in such testing apparatus.

The present invention seeks to overcome or mitigate the above problems associated with the prior art.

According to an aspect of the present invention, there is provided a method for testing a patient's visual field using testing apparatus having a display and a patient input device, the method comprising the steps of:

a) presenting a home target on the display;

b) displaying a cursor on the display, the cursor being moveable by the patient using the patient input device;

c) once the cursor is moved to the home target, presenting a visual field target on the display at a first position;

d) recording the visual field target as detected if the patient moves the cursor a predetermined distance towards the visual field target; and

e) repeating steps c) and d) for further visual field targets at further positions for determining the patient's visual field based on the positions of the visual field targets that have been detected.

In this way, the present invention provides a method for testing a patient's visual field that is more comfortable and interactive for the patient, which leads to visual field test results that are more reliable and more accurate.

In this connection, with the present invention, the requirement of conventional tests for a patient to rigidly maintain focus on a fixed fixation target for the duration of the visual field test is removed. Instead, the patient is initially tasked with moving the cursor to the home target, such that it is at least partially in contact with the home target. In doing this, the patient's focus is naturally drawn to the cursor, and consequently is directed at the home target when the cursor reaches the home target. The movement of the cursor over the home target then triggers the presentation of a visual field target at a first known position on the display. If the patient sees the presented target, the patient is prompted to move the cursor in the direction of the presented target. However, rather than requiring the patient to move the cursor the whole way to the target, which would be time consuming, once the patient has moved the cursor a predetermined distance from the home target toward the presented visual field target, the presented target is recorded as detected. As such, the patient is only required to move the cursor a short distance in the direction of the presented target in order to register their detection. The patient is then prompted to move the cursor back to the home target, which triggers continuation of the visual field test, with the presentation of the next visual field target at a further position on the display.

In view of the above, it will therefore be understood that the home target effectively serves as a centre of field target in that it designates the centre of the patient's field to be tested. That is, the patient is prompted to focus their gaze back to the home target at the start of each iteration of the test sequence through the requirement for them to move the cursor back to the home target. As such, the home target acts as the centre of the notional testing area, albeit that this centre does not necessarily need to correspond to the centre of the display since the position of the home target may itself be varied.

Advantageously, because the patient is not required to constantly focus on a centre of field target, the patient's experience of the visual field test is more comfortable and less strenuous. In essence, the test functions like an interactive game in which the patient's responses prompt progression through the sequence of presented visual field targets, thereby helping to maintain their concentration. These factors reduce the patient's tendency to look around at other areas of the display. As such, there is less chance of the patient inadvertently detecting a presented visual field target that is outside of their actual visual field. There is also less chance of the patient's peripheral vision becoming blurred, which can result in visual field targets not being detected by the patient. Thus, with the present invention, due to the above advantages, false positive and false negative responses by the patient are reduced, and therefore the accuracy and reliability of a visual field test is improved.

Preferably, the home target is presentable at different positions on the display. In this way, the home target can be repositioned on the display, either as part of a testing program or by a practitioner giving the visual field test to the patient. This effectively expands the size of the notional testing area, and hence the size of the visual field that can be tested with a given device. For example, by moving the home target to a position at the lower right of the display, the upper left part of the patient's visual field can be tested at a greater extremity. Consequently, even when using a display which provides a relatively narrow field of view, a practitioner is able to conduct a more expansive overall visual field test by carrying out a program of sub-tests directed to different regions of the larger combined notional testing area. That is, the home target can be repositioned at different sides or corners of the display screen in each sub-test so that the opposite extremity on the display is relatively further away so that a greater field of view is tested. As a result, the present invention allows for the use of much smaller displays for visual field testing, such as head mounted displays, which might otherwise provide too narrow a field of view. In turn, this avoids the requirement of conventional tests for specialist testing equipment.

Preferably, the patient input device is a computer mouse. Alternatively, the patient input device may be a joystick, touch screen, or drawing tablet and pen.

Preferably, in step c) the visual field target is presented once the cursor is moved at least partially over the home target. More preferably, in step c) the visual field target is presented once the cursor is moved fully over the home target. In this way, the patient is required to concentrate their focus on the cursor to accurately move it over the home target. As such, there is less chance of the patient preemptively looking for a new target.

Preferably, the predetermined distance is a distance from the home target. In this way, the patient's focus and their movement of the cursor is centred around the home target.

Preferably, the predetermined distance is defined by a boundary around the home target. Preferably, step d) comprises recording the visual field target as detected if the cursor is moved to the boundary in a direction towards the visual field target. More preferably, step d) comprises recording the visual field target as detected if the cursor is moved into contact with the boundary in a direction towards the visual field target. Preferably, the cursor is only moveable on the display within the boundary. In this way, the boundary provides a set perimeter surrounding the home target for designating the extent to which the patient is required to move the cursor.

Preferably, the boundary is circular. In this way, the boundary is equidistant in a radial direction from the home target so that the cursor is moved the same distance from the home target to the boundary, regardless of the position on the display of the presented visual field target.

Preferably, it is determined that the cursor has moved in a direction towards the visual field target if the cursor is moved into contact with a target region of the boundary for that respective visual field target, the target region comprising the point of intersection between the boundary and a path between the home target and the visual field target. In this way, a notional linear path between the home target and each presented visual field target is used to define a target region of the boundary for that particular visual field target. This designates a direction from the home target as being towards the presented visual field target. As the position of the boundary also defines the predetermined distance required for detection, the act of the cursor being moved to intersect with the target region of the boundary thereby indicates that the movement vector for the cursor meets the requirement for recording the visual field target as detected. Conversely, contact between the cursor and other regions of the boundary which are not target regions for the particular visual field target can then be identified as movements that are not in its direction, and hence the presented visual field target has not been seen.

Preferably, the target region extends circumferentially around a portion of the point of intersection. Preferably, the target region extends symmetrically about the point of intersection. In this way, the target region extends over a sufficient portion of the boundary to account for minor imprecision in movement of the cursor. That is, a patient may attempt to move the cursor directly towards the presented visual field target, but in practice this is unlikely to follow a precise linear path between the two. As such, the target region provides what is deemed to be an acceptable tolerance in the patient's control over the cursor that is still be deemed to be demonstrating a movement towards a detected visual field target.

Preferably, the boundary is displayed to the patient. In this way, the patient can see the distance from the home target that the cursor must be moved for detection, thereby helping them to control and limit cursor movement.

Preferably, the method further comprises the step of recording the visual field target as not detected if the cursor is moved into contact with a portion of the boundary that is not the target region for that respective visual field target. This may be recorded as a false positive.

Preferably, the method further comprises the step of recording the visual field target as not detected if, after a period of time, the cursor has not been moved in response to the presentation of a visual field target.

Preferably, the further visual field targets are presented in random or pseudo-random positions. In this way, it is possible to test different areas of the patient's visual field, without the patient recognising or learning a pattern in the sequence of displayed targets.

Preferably, the display is a head mounted display or retinal display unit for projecting images into a patient's eye or eyes, for example using digital light processing technology. In this way, a compact display is provided, rather than needing to provide a large display screen or specialised wide angle display apparatus. Furthermore, a retinal display unit allows the visual display test to easily be conducted on only one eye of the patient at a time, simply by only delivering light to the eye being tested.

Preferably, the method further comprises the step of preparing a visual field map based on the visual field targets that have been recorded as detected.

Preferably, at least one of a visual, auditory, or tactile response is emitted when the cursor is moved the predetermined distance. More preferably, at least one of a visual, auditory, or tactile response is emitted when a visual field target is recorded as detected. In this way, the patient is prompted to move the cursor back to the home target when the cursor has reached the boundary.

In an embodiment, the visual characteristics of each visual field target vary depending on the position of the visual field target on the display, the visual characteristics comprising at least one of the size, shape, colour and/or contrast. Preferably, the visual characteristics of each visual field target are varied to compensate for variations in sensitivity over a patient's field of view relative to the position of the visual field target on the display. Accordingly, this allows the present invention to compensate for reduced sensitivity at the periphery of the retina, for example by making the visual field targets larger, or brighter. Equally, to compensate for refractive distortions at a far periphery of the patient's field of view, a normally circular visual field target may, for example, be formed with an oval shape or near oval shape so that it still appears circular to the patient.

Preferably, the step of presenting a visual field target on the display comprises increasing the brightness of the visual field target and/or the contrast of the visual field target relative to the background display over a period of time. In this way, the contrast of the presented visual field target increases relative to the display to make it progressively easier for a user to detect the target.

Preferably, step d) further comprises recording the brightness and/or contrast level of a visual field target at the time of detection. In this way, it can be determined if there is a threshold level of visibility for visual field targets in different regions of a patient's visual field. Preferably, the step of recording the brightness and/or contrast level of a visual field target at the time of detection comprises accounting for the reaction time of the patient. That is, the recorded threshold contrast level would be the contrast level at the time when the target was recorded as seen, minus the reaction time taken to respond. In this way, a more accurate measurement of the brightness or contrast level at which a patient sees a target is recorded.

According to a further aspect of the present invention, there is provided a software program having instructions for implementing a method for testing a patient's visual field using testing apparatus having a display and a patient input device, the software program comprising:

a) instructions for presenting a home target on the display;

b) instructions for displaying a cursor on the display, the cursor being moveable by the patient using the patient input device;

c) instructions for, once the cursor is moved to the home target, presenting a visual field target on the display at a first position;

d) instructions for recording the visual field target as detected if the patient moves the cursor a predetermined distance towards the visual field target; and

e) instructions for repeating steps c) and d) for further visual field targets at further positions for determining the patient's visual field based on the positions of the visual field targets that have been detected. The software program may be provided stored on a non-transient computer readable medium containing the program instructions, which, when executed by a computer, implement the claimed method for testing a patient's visual field using the testing apparatus.

According to a further aspect of the present invention, there is provided apparatus for testing a patient's visual field, the apparatus comprising:

a display;

a patient input device;

a control unit configured to (a) present a home target on the display; (b) display a cursor on the display, the cursor being moveable by the patient using the patient input device; and (c) present, once the cursor is moved to the home target, a visual field target on the display at a first position; and

a memory for recording the visual field target as detected if the patient moves the cursor a predetermined distance towards the visual field target,

wherein the control unit is further configured to display further visual field targets at further positions and the memory is for recording the further visual field targets as detected if the patient moves the cursor a predetermined distance towards the respective visual field target.

According to a further aspect of the present invention, there is provided a peripheral visual field testing method, comprising: providing a centre of field target and a user controllable cursor on a display; presenting a test sequence of peripheral target spots at different positions on the display, where each peripheral target spot in the sequence is presented in response to the cursor being moved to the centre of field target; and recording user responses to the presented peripheral target spots for mapping the user's visual field, wherein a boundary is defined around the centre of field target, and a peripheral target spot is recorded as being seen by the user if the cursor is moved from the centre of field target such that it intersects the boundary at a position thereon that designates a direction of movement along a path toward the respective peripheral target spot.

According to a further aspect of the present invention, there is provided a peripheral visual field testing apparatus, comprising:

a display for displaying a centre of field target and a user controllable cursor;

a controller for presenting a test sequence of peripheral target spots at different positions on the display, where each peripheral target spot in the sequence is presented in response to the cursor being moved to the centre of field target; and

a memory for recording user responses to the presented peripheral target spots for mapping the user's visual field,

wherein a boundary is defined around the centre of field target, and a peripheral target spot is recorded as being seen by the user if the cursor is moved from the centre of field target such that it intersects the boundary at a position thereon that designates a direction of movement along a path toward the respective peripheral target spot.

Illustrative embodiments of the present invention will now be described with reference to the accompanying drawings, of which:

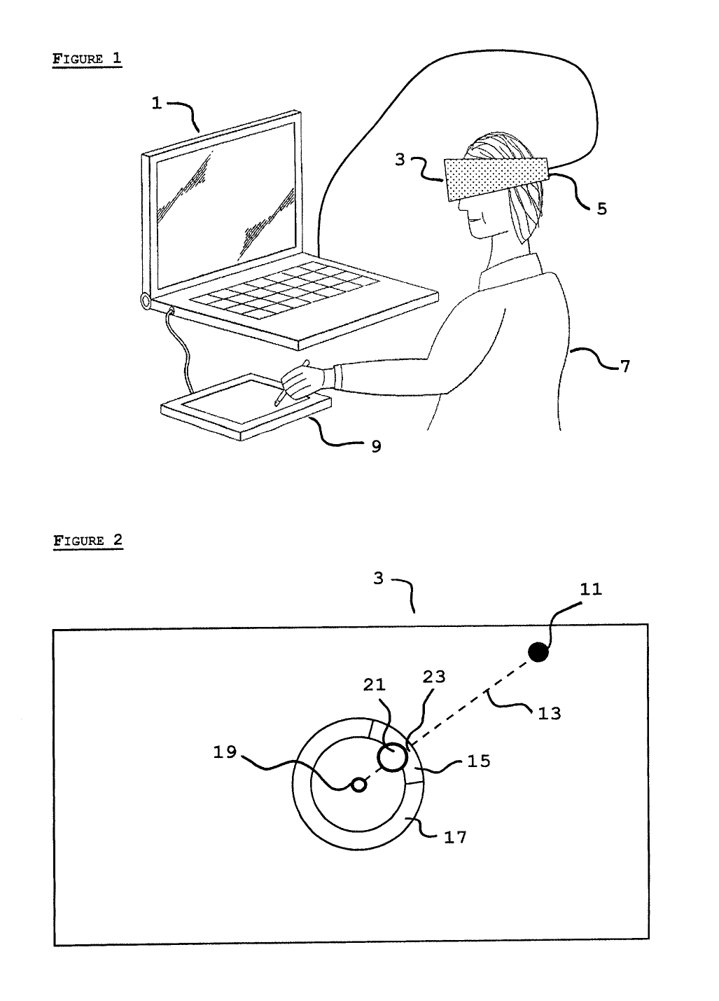

FIG. 1 shows a perspective view of apparatus for implementing an embodiment of the present invention;

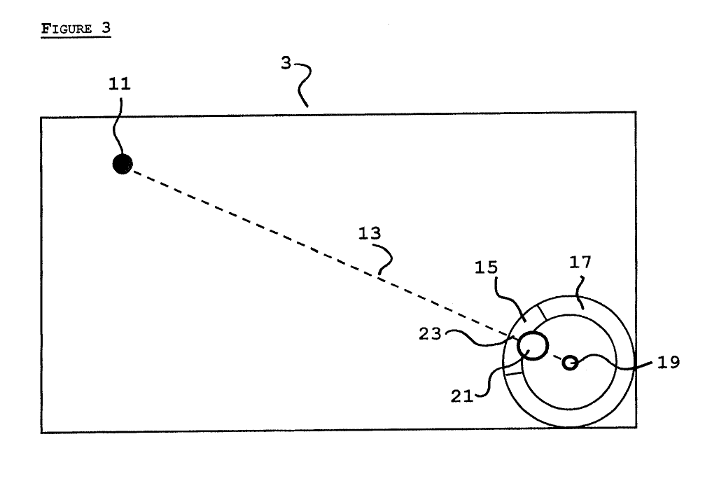

FIG. 2 shows a schematic representation of a visual field test display during a visual field test of an embodiment of the invention; and

FIG. 3 shows a schematic representation of a visual field test display during a visual field test of an alternative embodiment of the invention, where the home target has been placed at a different position on the display.

FIG. 1 shows an example of testing apparatus for implementing an embodiment of the present invention. As shown, the patient 7 is provided with a display 3 and a patient input device 9 connected to a computer 1. In this embodiment, the display 3 is part of a head mounted display unit 5 worn by the patient 7. The head mounted display unit 5 comprises a retinal display unit that projects light directly into a patient's eye or eyes such at an image of the visual field test display screen is formed on their retina. The computer 1 in this embodiment is a laptop computer on which is stored software with instructions for implementing the testing methodology according to this embodiment of the present invention. In other embodiments, the computer may be, for example, incorporated into the head mounted display unit 5. The patient input device 9 is a drawing tablet and pen. The display of the laptop computer 1 is not used by the patient during the visual field test, but may be used by a practitioner to administer test or monitor functions during the testing process.

In alternative embodiments of the invention, alternative displays 3 may be used. For example, more conventional head mounted displays which comprise small OLED, LED or LCD display screens may be used. The head mounted display may also be an actual flat or curved display in front of the patient's eye/s. Equally, more conventional large display screens, such as computer monitors, projector systems, or flat screen displays could also be used. In alternative embodiments, rather than using a pen and tablet, different patient input devices 9 may also be used, such as a computer mouse, joystick, or touch screen.

FIG. 2 shows a schematic representation of a visual field test screen similar that which would be visualised as being displayed on the display 3 to the patient 7 wearing the head mounted display unit 5. The display 3 has a display background on which a circular home target spot 19 is displayed, in this case at a position in the centre of the display 3. In alternative embodiments, such as that shown in FIG. 3, the home target spot 19 can be positioned at different locations on the display 3, as is discussed in further detail below.

A circular cursor 21 is also displayed and is moveable by the patient 7 by operating the patient input device 9.

A circular boundary 17 is also defined around the home target 19, set at a predetermined radial distance therefrom. For illustrative purposes, the circular boundary 17 is shown as displayed to the patient, although in alternative embodiments, the boundary 17 may be invisible to the patient, or only temporarily appear when contacted by the cursor 21.

In this embodiment, the display 3 provides the patient 7 with a field of view that extends 90 degrees horizontally and 40 degrees vertically. It will be appreciated that different displays will provide different maximum fields of view, depending on the size of the screen and the position of the patient's eye relative to the screen.

In use, one eye of the patient is typically tested at a time. In this embodiment, since the display 3 is part of a head mounted display unit 5 comprising a retinal display unit, in order to test only one of the patient's eyes, the visual field test is simply only projected into one of the patient's eyes. In alternative embodiments, the vision of the patient's other eye may be physically blocked.

At the start of a test program, the home target spot 19 and moveable cursor 21 is displayed on display 3. The patient is prompted to begin the test by moving the cursor 21 to the home target spot 19 using the user input device 9. The patient is also instructed to move the cursor from the home target 19 towards any presented visual field target they subsequently see. These prompts may be from a practitioner administering the test, or be provided automatically by the software generating audio or on-screen instructions.

In response to the cursor 21 being moved into contact with the home target spot 19, the software generates a first visual field target 11 on the display 3 at a first known position.

The presentation of the first visual field target 11 establishes a notional linear path 13 between the home target 19 and the presented visual field target 11 along which the cursor 21 would travel if it were moving directly to the visual field target 11. This path is not visible to the patient 7. The point of intersection 23 between the boundary 17 and the path 13 is used to define a target region 15 on the boundary 17 for that particular presented visual field target 11. As such, the target region 15 designates a direction of travel for the cursor which is deemed to be heading towards the presented visual field target. The target region 15 extends circumferentially around a portion of the point of intersection 23, and extends symmetrically either side of this. This provides an allowance or tolerance for deviations of the cursor 21 from the notional linear path 13.

If the patient 7 detects the presented visual field target 11, they will move the cursor 21 towards it in accordance with the pre-test instructions. Once the cursor 21 has travelled the distance set by the boundary 17, it will intersect with it. If this contact is within the target region 15 of the boundary 17, it is determined that the visual field target 11 has been seen and hence its displayed position is within the patient's visual field. The patient 7 is then notified that this positive detection has been recorded by a visual or auditory response signal, and the visual field target 11 will then disappear.

If the patient 7 does not move the cursor 21 on presentation of a visual field target 11, after a period of time, the visual field target 11 will be recorded as undetected and will then disappear. In alternative embodiments, if visual field targets 11 remain undetected for a period of time, their size and/or brightness and/or contrast may be increased for a period of time before recording them as undetected.

If, on presentation of a visual field target 11, the patient 7 moves the cursor 21 from the home target 19 into contact with a section of the boundary 17 other than the target region 15, the visual field target 11 will be recorded as undetected since this would represent an erroneous response. The visual field target 11 will then disappear and a visual or auditory response is provided to prompt the patient to move the cursor 21 back into contact with the home target 19.

Once a visual field target is recorded as seen or not seen, it disappears from the display screen and the visual or auditory response prompts the patient to move the cursor 21 back into contact with the home target spot 19. This causes the display of the next visual field target in the test sequence. This establishes a new target region 15 of the boundary 17 based on the relative position of that newly displayed visual field target since, typically, the new visual field target will be in a different position on the display 3. That said, in some circumstances, the software may generate a new visual field target in the same position as a previous target, for example, if it is identified that a particular area of the patient's visual field should be re-tested.

The above steps are repeated until a number of patient responses have been recorded for a number visual field targets at various positions across the display. As such, the positions of the presented sequence of visual field targets 11 and associated recorded patient responses may be used to generate a visual field map. This can show regions which do not appear visible to a patient and hence help identify defects in their vision.

FIG. 3 shows schematic representation of a visual field test similar to that shown in FIG. 2, except that in this embodiment the home target has been positioned at the lower right corner of the display 3.

In this respect, it will be understood that the home target spot 19 may be moved to any position on the display 3, and FIG. 3 simply shows one alternative position. This movement may be under the control of a practitioner or may be part of wider visual field testing program.

In this connection, the same features are present in FIG. 3 as those described above with reference to FIG. 2 and the operation of the test itself is the same. However, by conducting visual field tests with the home target 19 in different positions on the display 3, it is possible to expand the effective field of view being tested. For example, by moving the home target 19 to the lower right of the display 3, a wider range of the upper left part of the patient's visual field can be tested.

To explain the above further, as mentioned above, the display 3 in these embodiments provides the patient 7 with a field of view that extends 90 degrees horizontally and 40 degrees vertically relative to the centre of their focus. Consequently, in the case of FIG. 2 where the home target 19 is in the centre of the display screen 3, with the patient 7 focussing on this, the maximum field that can be tested is 45 degrees in a horizontal axis left or right, and 20 degrees in a vertical axis up or down. However, with the home target 19 moved to the bottom right corner of the display 3 in FIG. 3, the field extending to the left and up is made relatively large. For instance, if we say FIG. 3 shows the home target 19 is positioned at 40 degrees right of the centre of the display and 15 degrees down of the centre of the display, with the patient's eye focussed here, the field of view which can be tested is up to 85 degrees left and 35 degrees up. The results from multiple visual field tests, with the home target in different positions can then be combined to produce an enlarged visual field test over an expanded range of a patient's visual field. For instance, in the above example, four sub-tests of sequences of targets could be conducted with the home target positioned in each corner of the display. This would then provide an effective visual field test area of 170 degrees horizontally and 70 degrees vertically.

Repositioning of the home target 19 is particularly useful with displays that only achieve a narrow field of view. For example, smaller display screens and head mounted display units had not previously been considered for visual field testing because the maximum field of view provided is relatively narrow.

Accordingly, the present invention provides a method for testing a patient's visual field that is more comfortable and interactive for the patient, and hence leads to visual field test results that are more reliable and more accurate. At the same time, the speed of the test is improved by only requiring the patient to move their cursor a short distance towards a target, rather than the whole way. Finally, because of the way that cursor movement and a patent's focus is centred on the home target, repositioning the home target allows the present invention to be implemented on a display which is only capable of providing a relatively narrow field of view, whilst still achieving testing results over a much larger effective area. As a result, the invention may be implemented on more compact and less specialised equipment, thereby reducing many of the barriers in terms of cost and space which traditionally prevent clinical practices from purchasing their own visual testing apparatus.

It will be understood that the embodiment illustrated above shows applications of the invention only for the purposes of illustration. In practice the invention may be applied to many different configurations, the detailed embodiments being straightforward for those skilled in the art to implement.

For example, the above illustrative examples have described the invention in terms of the testing method, but it will be understood that these methods may be implemented using a software programme with instructions for implementing the method steps. Equally, testing apparatus may be provided which operates under control of such software for implementing embodiments of the invention.

Furthermore, in embodiments, if a presented visual field target 3 is not recorded as seen by a patient 7, it may be displayed again, before a negative response is recorded.

Furthermore, the reaction time of the patient may be recorded. That is, the time taken between presentation of the visual field target 11 and movement of the cursor 21 to the target region 15 of the boundary 17 by the patient 7 may be recorded. Moreover, the movement time taken by the patient to actually move the cursor from the home target 19 to the target region 15 of the boundary 17 may be subtracted from the measured reaction time to give a true response time. That is, the time between presentation of the target 11 and movement of the cursor off of the home target 19, which subsequently leads to a positive detection. Alternatively, reaction time could be measured by presenting a cursor target next to, but separate from the home target 19, and recording the time between the cursor 21 contacting this target and contacting the target region 15 of the boundary 17.

Moreover, the level of contrast between the presented visual field target 11 and the display background may be recorded when a visual field target 11 is recorded as detected. For example, in embodiments, a visual field target 11 may be presented by fading in and becoming progressively more visible. That is, visual field target 11 may start from a point where it is substantially blended with the display background, with a similar colour and brightness. The contrast with the background may then increase steadily over a period of time until the visual field target is detected or a maximum contrast or brightness is reached. With such embodiments, the recorded movement time leading to a positive detection may then be used to determine the contrast level of the target 11 when it was detected by the patient 7. This can provide an indication of a threshold level of visibility for targets on the results map.

In addition, a practitioner may observe the patient's eye during the test using a video camera feed. This may allow the practitioner to notify the patient 7 when they are not looking at the cursor 21 or the practitioner may be provided with a switch for recording periods in which the patient's fixation on the cursor was lost and thereby results recorded during such periods may be recorded as invalid. Alternatively, an eye tracker may be provided for tracking the eye being tested and stopping the test when it detects the eye gaze has moved from the cursor.

In addition, although in the above example only one eye has been tested at a time, in other embodiments both of the patient's eyes could be tested simultaneously.

1. A method for testing a patient's visual field using a testing apparatus having a display and a patient input device, the method comprising the steps of:
a) presenting a home target on the display;
b) displaying a cursor on the display, the cursor being moveable by the patient using the patient input device;
c) once the cursor is moved to the home target, presenting a visual field target on the display at a first position;
d) recording the visual field target as detected if the patient moves the cursor a predetermined distance towards the visual field target; and
e) repeating steps c) and d) for further visual field targets at further positions for determining the patient's visual field based on the positions of the visual field targets that have been detected.

2. A method according to claim 1, wherein the home target is presentable at different positions on the display.

3. A method according to claim 1, wherein in step c) the visual field target is presented once the cursor is moved at least partially over the home target.

4. A method according to claim 1, wherein the predetermined distance is a distance from the home target.

5. A method according to claim 1, wherein the predetermined distance is defined by a boundary around the home target.

6. A method according to claim 5, wherein the boundary is circular.

7. A method according to claim 5, wherein step d) comprises recording the visual field target as detected if the cursor is moved to the boundary in a direction towards the visual field target.

8. A method according to claim 7, wherein it is determined that the cursor has moved in a direction towards the visual field target if the cursor is moved into contact with a target region of the boundary for that respective visual field target, the target region comprising a point of intersection between the boundary and a path between the home target and the visual field target.

9. A method according to claim 8, wherein the target region extends circumferentially around a portion of the point of intersection.

10. A method according to claim 9, where the target region extends symmetrically about the point of intersection.

11. A method according to claim 8, further comprising the step of recording the visual field target as not detected if the cursor is moved into contact with a portion of the boundary that is not the target region for that respective visual field target.

12. A method according to claim 5, wherein the cursor is only moveable on the display within the boundary.

13. A method according to claim 5, wherein the boundary is displayed to the patient.

14. A method according to claim 1, further comprising the step of recording the visual field target as not detected if, after a period of time, the cursor has not been moved in response to the presentation of a visual field target.

15. A method according to claim 1, further comprising the step of preparing a visual field map based on the visual field targets that have been recorded as detected.

16. A method according to claim 1, wherein step d) further comprises recording a reaction time of the patient.

17. A method according to claim 1, wherein step c) of presenting the visual field target on the display comprises increasing at least one of a brightness of the visual field target and a contrast of the visual field target relative to a background over a period of time.

18. A method according to claim 17, wherein step d) further comprises recording at least one of the brightness and the contrast level of the visual field target at a time of detection.

19. A method according to claim 18, wherein the step of recording the brightness or the contrast level of a visual field target at the time of detection comprises accounting for a reaction time of the patient.

20. A non-transient computer readable medium containing program instructions for causing a computer to implement a method for testing a patient's visual field using a testing apparatus having a display and a patient input device, the program instructions comprising:
a) instructions for presenting a home target on the display;
b) instructions for displaying a cursor on the display, the cursor being moveable by the patient using the patient input device;
c) instructions for, once the cursor is moved to the home target, presenting a visual field target on the display at a first position;
d) instructions for recording the visual field target as detected if the patient moves the cursor a predetermined distance towards the visual field target; and
e) instructions for repeating steps c) and d) for further visual field targets at further positions for determining the patient's visual field based on the positions of the visual field targets that have been detected.

21. An apparatus for testing a patient's visual field, the apparatus comprising:
a display;
a patient input device;
a control unit configured to (a) present a home target on the display; (b) display a cursor on the display, the cursor being moveable by the patient using the patient input device; and (c) present, once the cursor is moved to the home target, a visual field target on the display at a first position; and
a memory for recording the visual field target as detected if the patient moves the cursor a predetermined distance towards the visual field target,
wherein the control unit is further configured to display further visual field targets at further positions and the memory is for recording each of the further visual field targets as detected if the patient moves the cursor the predetermined distance towards the respective one of the further visual field targets.
